(19) United States
(12) Patent Application Publication
(10) Pub. No.: US 2014/0271751 A1
(43) Pub. Date: Sep. 18, 2014

(54) PROCESS FOR ENCAPSULATING A HYDROPHOBIC ACTIVE

(71) Applicant: DOW GLOBAL TECHNOLOGIES LLC, Midland, MI (US)

(72) Inventors: Dale C. Schmidt, Midland, MI (US); David L. Malotky, Midland, MI (US)

(21) Appl. No.: 14/351,204

(22) PCT Filed: Oct. 16, 2012
(86) PCT No.: PCT/US2012/060374

§ 371 Date: Apr. 11, 2014

Related U.S. Application Data
(60) Provisional application No. 61/548,854, filed on Oct. 19, 2011.

(51) Int. Cl. A61K 8/11; A61Q 17/04

(52) U.S. Cl. CPC A61K 8/11; A61Q 17/04
USPC 424/401; 424/59

(57) ABSTRACT

Described are processes for encapsulating a hydrophobic active in a core-shell mesocapsule comprising preparing a high internal phase ratio emulsion having a continuous aqueous phase and a dispersed oil phase comprising at least one hydrophobic active and one or more pre-polymers, reducing the volume fraction of the high internal phase ratio emulsion below 0.74 with an aqueous phase stream, and then forming a dispersion of core-shell mesocapsules containing hydrophobic active by either: i) allowing the reduced volume fraction emulsion to sit for 12 hours when the pre-polymer is isocyanate, or ii) contacting the reduced volume fraction emulsion with a third aqueous stream comprising a cross-linking agent.

### FIELD

The invention relates generally to processes for encapsulating a hydrophobic active and in particular to a hydrophobic active encapsulated in a core-shell mesocapsule.

### BACKGROUND

High internal phase ratio (HIPR) emulsions are known to be useful for efficiently incorporating hydrophobic actives (for example, sunscreens) into cosmetic formulations. As might be expected, such emulsions are characterized by having relatively high solids, low amount of solvent or external phase present (high internal phase).

Some hydrophobic actives are better applied in an encapsulated form, for example to prevent skin irritation, sensitization, or control their release into a formulation. One way to encapsulate hydrophobic actives is through interfacial polymerization, i.e., a reaction between two organic intermediates that takes place at an interface between two immiscible liquids (one immiscible liquid is dispersed in the other immiscible liquid). A “core-shell” capsule is formed around the dispersed phase. However, inter facial polymerization requires a very dilute reaction medium in order to promote formation of discrete encapsulated particles as opposed to agglomeration, which would occur at higher concentrations. Accordingly, one skilled in the art faced with the need make a cosmetic emulsion and encapsulate a hydrophobic active would logically consider interfacial polymerization and HIPR to be mutually exclusive.

Therefore, what is needed are improved processes for encapsulating a hydrophobic active, and in particular, to a hydrophobic active encapsulated in a core-shell mesocapsule.

### DETAILED DESCRIPTION

In one embodiment, the present invention provides a process for encapsulating a hydrophobic active in a core-shell mesocapsule comprising preparing a high internal phase ratio emulsion having a continuous aqueous phase and a dispersed oil phase comprising at least one hydrophobic active and one or more pre-polymers, reducing the volume fraction of the high internal phase ratio emulsion below 0.74 with an aqueous phase stream, and then forming a dispersion of core-shell mesocapsules containing hydrophobic active by either i) allowing the reduced volume fraction emulsion to sit for 12 hours when the pre-polymer is isocyanate, or ii) contacting the reduced volume fraction emulsion with a third aqueous stream comprising a cross-linking agent.

Without being bound by theory, it is believed that an interfacial polymerization reaction involving the one or more pre-polymers at interface between the aqueous phase and the oil phase takes place, forming a polymeric shell and thereby encapsulating the at least one hydrophobic active in the core-shell mesocapsule.

As used herein, the term “pre-polymer” includes a compound, a monomer, a polymer or any combinations thereof which can undergo interfacial polymerization reaction to form the polymeric shell. In one embodiment, the pre-polymer comprises a reactive species such as an isocyanate which can further react with components of the aqueous phase, in particular water of the aqueous phase, to form a polyurea shell. In one embodiment, the one or more pre-polymers are present in the oil phase at no more than 20% by volume.

In one embodiment, the pre-polymer is an isocyanate or a mixture of isocyanates. Exemplary isocyanates include, but are not limited to, toluene diisocyanate (TDI), diisocyanato-diphenylmethane (MDI), derivatives of MDI such as polymethylene polyphenylisocyanate that contains MDI, an example of which is PAPI 27™ polymeric MDI (The Dow Chemical Company), isophorone diisocyanate, 1,4-diisocyanatobutane, phenylene diisocyanate, hexamethylene diisocyanate, 1,3-bis(isocyanatomethyl)benzene, 1,8-disocyanatooctane, 4,4′-methylenebis(phenyl isocyanate), 4,4′-methylenebis(cyclohexyl isocyanate) and mixtures thereof. In a preferred embodiment the isocyanate is a polyisocyanate selected from a group of 4,4′-diisocyanatodiphenylmethane, 2,4′-diisocyanatodiphenylmethane, p-phenylene diisocyanate, 2,6-toluene diisocyanate, polyphenyl polymethylene polyisocyanate, 1,3-bis(isocyanatomethyl)cyclohexane, 1,4-diisocyanatocyclohexane, hexamethylene diisocyanate, 1,5-naphthalene diisocyanate, 3,3′-dimethyl-4,4′-biphenyl diisocyanate, 4,4′-diisocyanatodicyclohexylmethane, 2,4′-diisocyanatodicyclohexylmethane, isophorone diisocyanate, or 2,4-toluene diisocyanate, or a combination thereof. In some embodiments, the one or more pre-polymers comprise a diacid chloride, a polyacid chloride, a sulfonyl chloride, a chloroformate or any mixtures thereof.

The term “hydrophobic active” refers to a personal care, fabric or surface care, or an agricultural active ingredient, that has a solubility in water of less than 100 ppm, more preferably less than 10 ppm. Alternatively, if a fragrance is encapsulated, the solubility in water may be less than 1000 ppm and still within the scope of the disclosure.

Personal care hydrophobic actives include emollients, moisturizers, fragrances, vitamins, anti-aging actives, and sunscreens typically used in personal care compositions in amounts of which falls within the regulatory approved limits In a preferred embodiment, the personal care agent is a sunscreen agent. Examples of sunscreen agents include, but are not limited to, p-aminobenzoic acid as well as salts and esters thereof; o-aminobenzoic acid and o-aminobenzoates (including methyl, menthyl, phenyl, benzyl, phenylethyl, linalyl, terpinyl, and cyclohexenyl esters thereof); salicylic acid and salicylates (including octyl, amyl, phenyl, benzyl, menthyl, glyceryl, and dipropyleneglycol esters thereof); cinnamic acid and derivatives thereof (including methyl and benzyl esters, alkyl alkoxycinnamates such as octyl methoxycinnamate (also known as 2-ethylhexyl-4-methoxycinnamate), alpha-phenyl cinnamonitrile, and butyl cinnamoyl pyruvate); dihydroxycinnamic acid and its derivatives; trihydroxycinnamic acid and its derivatives; diphenylbutadiene and stilbene; dibenzalacetone and benzalacetophenone; I naphthosulfonates (such as sodium salts of 2-naphthol-3,6-disulfonic acid and 2-naphthnol 6, 8-disulfonic acid); dihydroxynaphthoic acid and its salts; o-and p-hydroxydiphenyldisulfonates; coumarin and derivatives thereof (such as 7-hydroxy, 7-methyl, and 3-phenyl coumarin); diazoles; quinine salts; quinoline and derivatives thereof; hydroxy-or alkoxybenzophenones; uric and vilouric acids; tannic acid and derivatives thereof; hydroquinone; benzophenones (such as oxybenzone, sulisobenzone, dioxybenzone, benzoresorcino1,2,2′,4,4′-tetrahydroxybenzophenone, 2,2′-dihydroxy-4, 4′ dimethoxybenzophenone, octabenzone, 4-isopropyldibenzoylmethane,-4-butylmethoxydibenzoylmethane, etocrylene, and 4-isopropyl-dibenzoylmethane), and mixtures thereof. In some embodiments, the sunscreen agents include ethylhexyl salicylate, homosalate, butyl methoxydibenzoylmethane, octocrylene, phenylbenzimidazole sulfonic acid, benzophenone-3, benzophenone-4, benzophenone-5, n-hexyl 2-(4-diethylamino-2-hydroxybenzoyl) benzoate, 4-methylbenzylidene camphor, terephthalylidene dicamphor sulfonic acid, disodium phenyl dibenzimidazole tetrasulfonate, methylene bis-benzotriazolyl tetramethylbutylphenol, bis-ethylhexyloxyphenol methoxyphenyl triazine, ethylhexyl triazone, diethylhexyl butamido triazone, 2,4,6-tris(dineopentyl 4′-aminobenzalmalonate)-s-triazine, 2,4, 6-tris (diisobutyl 4′-aminobenzalmalonate)-s-triazine, 2,4-bis(n-butyl 4′-aminobenzoate)-6-(aminopropyltrisiloxane)-s-triazine, 2,4-bis(dineopentyl 4′-aminobenzalmalonate)-6-(n-butyl 4′-aminobenzoate)-s-triazine, 2,4, 6-tris(biphenyl-4-yl)-1,3,5-triazine, 2,4,6-tris(terphenyl)-1,3,5-triazine, drometrizole trisiloxane, polysilicone-15, 1,1-dicarboxy (2,2′-dimethylpropyl)-4,4-diphenylbutadiene, 2,4-bis [5-1-(diraethylpropyl) benzoxazol-2-yl (4-phenyl) imino]-6-(2-ethylhexyl)-imino-1,3,5-triazine, and any mixtures thereof. In one embodiment, the sunscreen agent is paraminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide and zinc oxide, diethanolamine methoxycinnamate, digalloy trioleate, ethyl dihydroxypropyl PABA, glyceryl aminobenzoate, lawsone with dihydroxy acetone, red petrolatum, and any combinations thereof. In one preferred embodiment, the sunscreen agent is octyl methoxycinnamate, in another preferred embodiment the sunscreen agent is avobenzone.

Other actives include triclosan, polyphenols, flavonoids and isoflavonoids, coenzyme Q10 (CoQ10) and derivatives thereof, carotene and derivatives thereof, salicylic acid and derivatives thereof, dehydroepiandrosterone (DHEA), hydrophobic polysaccharides, proteins, including enzymes and peptides, and botanicals. Exemplary vitamins include Vitamin A and esters thereof, Vitamin D and derivatives thereof, Vitamins B3 and B5 and derivatives thereof, Vitamin E and esters thereof, Vitamin F and derivatives thereof, and Vitamin K.

In one embodiment, the hydrophobic active is a fragrance oil. Examples include scents that are floral, ambery, woody, leather, chypre, fougere, musk, mint, vanilla, fruit, and/or citrus. Fragrance oils are obtained by extraction of natural substances or synthetically produced. In one embodiment, the fragrance oil is one or more of an essential oil.

The term “agricultural active ingredient” as used herein refers to an active used in agriculture, horticulture and pest management for protection of crops, plants, structures, humans and animals against unwanted organisms such as fungal and bacterial plant pathogens, weeds, insects, mites, algae, nematodes and the like. Specifically, active ingredients used for these purposes include fungicides, bactericides, herbicides, insecticides, miticides, algaecides, nemtocides and fumigants. The term “agricultural active ingredient” also includes insect attractants, repellants and pheromones, modifiers of plant physiology or structure and herbicide safeners.

In one embodiment of the invention, the process for preparing HIPR emulsion comprises in the presence of an emulsifying and stabilizing amount of a surfactant, mixing a first aqueous phase stream and an oil phase stream comprising at least one hydrophobic active and one or more pre-polymers in a first mixer. The surfactant can be in either the phase, and suitable surfactants include nonionic, anionic, cationic, or combinations of nonionic and anionic or nonionic and cationic. Examples of suitable surfactants include alkali metal lauryl sulfates such as sodium dodecyl sulfate, alkali metal fatty acids salts such as sodium oleate and sodium stearate, alkali metal alkylbenzene sulfonates such as sodium dodecylbenzene sulfonate, polyoxyethylene nonionics, and quaternary ammonium surfactants.

High internal phase ratio (HIPR) emulsions are characterized, in general, by having a volume fraction of greater than 0.74, up to 0.99. The aqueous phase stream may pass through an inlet at a flow rate of R1 and the oil phase stream introduced through another inlet at a flow rate of R2, adjusted to form an HIPR emulsion having the desired particle size and polydispersity of the intended application. In one embodiment, the ratio of R1 to R2 is 20:80 to 5:95. In another preferred embodiment, the ratio of R1 to R2 is 10:90.

The term “particle size” of the mesocapsule is defined as the volume average diameter (Dv) of the mesocapsule. In one embodiment, the volume average diameter of the core-shell mesocapsule is less than 1500 nanometers. In another embodiment, the volume average diameter of the mesocapsule is between 500 and 1500 nanometers. In yet another embodiment, the volume average diameter of the mesocapsule is between 30 and 500 nanometers. The term “polydispersity” as used herein, is defined as the ratio of the volume average diameter (Dv) and the number average diameter (Dn) of the particles, or Dv/Dn.

The first aqueous phase stream comprises water. In certain embodiments, the first aqueous phase stream can additionally include water soluble ingredients such as rheology modifiers, preservatives, humectants, pH modifiers, surfactants and mixtures thereof. Example ingredients include, but are not limited to, carbomers, acrylic copolymers, polyacrylamides, polysaccharides, natural gums, clays, alkyl esters of p-hydroxybenzoic acid, glycerol and mixtures thereof.

In certain embodiments, the hydrophobic active is a solid at room temperature and must be dissolved in a suitable solvent prior to providing in the oil phase stream. In one embodiment, a poorly water-soluble hydrophobic active is dissolved in a solvent that readily dissolves the active. Suitable solvents may be one or a mixture of organic solvents that have low water solubility, which includes, but are not limited to, petroleum fractions or hydrocarbons such as mineral oil, aromatic solvents, xylene, toluene, paraffinic oils, and the like; vegetable oils such as soy bean oil, rape seed oil, olive oil, castor oil, sunflower seed oil, coconut oil, corn oil, cotton seed oil, linseed oil, palm oil, peanut oil, safflower oil, sesame oil, tung oil and the like; esters of the above vegetable oils; esters of monoalcohols or dihydric, trihydric, or other lower polyalcohols (4-6 hydroxy containing), such as 2-ethyl hexyl stearate, ethylhexyl benzoate, isopropyl benzoate, n-butyl oleate, isopropyl myristate, propylene glycol dioleate, di-octyl succinate, di-butyl adipate, di-octyl phthalate, acetyl tributyl citrate, triethylcitrate, triethyl phosphate, and the like; esters of mono, di and polycarboxylic acids, such as benzylacetate, ethylacetate, and the like; ketones, such as cyclohexanone, acetophenone, 2-heptanone, gamma- butyrolactone, isophorone, N-ethyl pyrrolidone, N-octyl pyrrolidone, and the like; alkyldimethylamides, such as dimethylamide of C8 and C10, dimethylacetamide, and the like; alcohols of low water solubility (i.e. 10 g/100 ml or less) such as benzyl alcohol, cresols, terpineols, tetrahydrofurfurylalcohol, 2-isopropylphenol, cyclohexanol, n-hexanol, and the like. In some cases, an additive is added to the oil phase, ostensibly to preserve the stability of an emulsion that will be created later in the process when the oil phase is mixed with an aqueous phase. This additive is a highly water-insoluble material that 1) has a negligible diffusion coefficient and negligible solubility in the continuous aqueous phase and 2) is compatible with the dispersed phase. Examples of additives include long chain paraffins such as hexadecane, polymers such as polyisobutene such as, for example, Indopol™ HI5 (INESO Oligomers), polystyrene, polymethylmethacrylate, natural oils such as seed oils, and silicones such as silicone oil or dimethicone. Preferably, the additive is used in an amount not greater than 10 weight percent based on the weight of the dispersed phase.

The HIPR emulsion formed in the first mixer is introduced into a second mixer. In the second mixer, the HIPR emulsion is diluted by providing the second aqueous phase stream to form a diluted emulsion having a dispersed oil phase volume fraction of less than 0.74. In certain embodiments, the second aqueous stream may further contain one or more of surfactants and water soluble ingredients. Example ingredients include, but are not limited to, conventional rheology modifiers, thickening agents and preservatives. In one embodiment, the second aqueous phase stream is introduced in the second mixer at a flow rate of R3 and the third stream is at a flow rate of R4. In one embodiment, a ratio of R3 to R4 is 60:20.

The diluted emulsion is contacted with a third stream comprising the cross-linking agent to perform an interfacial polymerization reaction between the one or more pre-polymers and the cross-linking agent at an interface between the aqueous phase and the oil phase to form a polymeric shell thereby encapsulating the at least one hydrophobic active in the core-shell mesocapsule. The term “cross-linking agent” as used herein means a substance that initiates and facilitates reaction of pre-polymers to form a core-shell capsule.

The cross-linking agent becomes part of the polymer structure comprising the core shell mesocapsule. In one embodiment, the cross-linking agent includes a hydroxyl-containing or amine-containing compound. In one embodiment, the hydroxyl-containing compound comprises water. Exemplary amine-containing compound includes water-soluble diamines, ethylenediamine, water-soluble polyamines, diethylenetriamine, triethylenetetramine, tetraethylenepentamine, pentaethylenehexamine, water-soluble polyamino acids, L-lysine, arginine, histidine, serine, threonine, polymers or oligomers of the aminoacids, water-soluble diols, ethylene glycol, propylene glycol, polyethylene oxide diol, resorcinol, water soluble polyols, 2-aminoethanol, guanidine, guanidine compounds, polyamidines and derivatives and any mixtures thereof. In one embodiment, an isocyanate present in the phase stream reacts with an amine in the cross-linking agent stream to form a polyurea shell thus forming a mesocapsule. In certain embodiments, the cross-linking agent can further comprise other functionalities that can be built into the polymeric shell and at least partially exposed on the surface of the polymeric shell. In one embodiment the cross-linking agent comprises a diamine with a carboxylate functionality (such as L-lysine) which reacts to form a polyurea shell that includes carboxylate functional groups at the surface of the mesocapsule. This carboxylate functionality may be unneutralized or it may be partly or fully neutralized to form a carboxylate salt. Some of the other functional group includes, but are not limited to, salts of carboxylate, phosphonate, salts of phosphonate, phosphate, salts of phosphate, sulfonate, salts of sulfonate, quaternary ammonium, betaine, oxyethylene or oxyethylene-containing polymers.

According to embodiments of the invention, mesocapsules of desired particle size and polydispersity is achieved by interfacial polymerization reaction between components of the diluted emulsion and the third stream comprising the cross-linking agent. In one embodiment, diluted emulsion is obtained in the first mixer by maintaining larger volumes or flow-rates of second aqueous phase stream compared to the third stream comprising the cross-linking agent. In another embodiment, interfacial polymerization reaction between components of the diluted emulsion and the third stream comprising the cross-linking agent is accomplished by providing a time-lag between the introduction of second aqueous phase stream and the third stream in the second mixer thus minimizing or preventing premature interfacial polymerization reaction between components of the HIPR emulsion and the third stream. In one embodiment, the time-lag is achieved by introducing the third stream comprising the cross-linking agent after a time interval subsequent to the introduction of the second aqueous phase stream. In another embodiment, the time-lag is achieved by introducing the third stream and the second aqueous phase stream at different sections or locations within the second mixer that are separated in space thus creating time-lag. The time-lag advantageously allows for the HIPR emulsion to completely mix with the second aqueous phase stream.

The reaction parameters can be optimized to increase or decrease the interfacial polymerization reaction rate and thereby optimizing the property of the mesocapsule. These parameters include, for example, temperature, pH, flow rates of the streams, mixing rate, reaction times, osmotic pressure and changing the levels or amount and the type of the pre-polymers, cross-linking agent, and solvents in the second mixer.

Exemplary properties of the mesocapsule includes polymeric shell thickness. In one embodiment, the shell thickness is between 10 nanometers and 40 nanometers. In one embodiment, for mesocapsules having a volume average diameter of less than 4 micrometers, it is desirable to have shell thickness of greater than 10 nm.

The mesocapsules of the present disclosure can be used with many conventional formulation ingredients such as aqueous or non-aqueous solvent media or diluents in which the mesocapsules are suspended or slurried at a concentration of hydrophobic active, with respect to the formulation, from 0.1% to 30%. Conventional inactive or inert ingredients such as dispersants, thickening agents, stickers, film-forming agents, buffers, emulsifiers, anti-freeezing agents, dyes, stablizers, solid carriers and the like may also be incorporated into formulations containing mesocapsules.

### EXAMPLES

### Example 1

A dispersion of core-shell mesocapsules is formed according to one embodiment of the present invention as follows. An oil phase comprising 81.3% octinoxate, 11.8% polymeric isocyanate (PAPI 27), 3.0% laureth-4, 3.0% laureth-23, 0.9% tridecyl trimellitate is combined, heated to 65° C., and mixed until uniform. The oil phase stream is fed into a two-inch diameter Oakes rotor stator mixer spinning at 1200 rpm from a pressurized tank by a Zenith gear pump. In the mixer, the oil phase stream is combined with an aqueous phase stream containing deionized water to form a HIPR emulsion. The first aqueous phase stream is pumped with a 500D Isco Syringe Pump.

The HIPR emulsion is conveyed to a second two-inch diameter Oakes mixer spinning at 900 rpm where it is diluted with a second aqueous stream containing deionized water pumped using a 500D Isco syringe pump to form a reduced volume fraction emulsion (no longer a HIPR).

Next, the reduced volume fraction emulsion is combined with a third aqueous stream comprising a 10% weight solution of ethylene diamine as a cross linking agent using a 500D Isco syringe pump. The cross-linking agent is added at a slightly less than stoichiometric amount to ensure complete consumption.

Rate of oil phase stream: 10 g/min; rate of first aqueous stream: 1.2 g/min; Rate of second aqueous stream: 6.31 g/min; and rate of crosslinking agent: 2.49 g/min

### Example 2

A dispersion of core-shell mesocapsules is formed according to another embodiment of the present invention as follows. An oil phase comprising 41.0% avobenzone, 20.5% octocrylene, 20.5% homosalate, 12% polymeric isocyanate (PAPI 27), 3.0% laureth-23, and 3.0% ceteth-20 is heated to 60° C., and mixied until the phase is uniform. The oil phase stream is fed to a four-inch diameter Oakes rotor stator mixer spinning at 620 rpm from a pressurized tank by a Zenith gear pump. In the mixer, the oil phase stream is combined with a first aqueous phase stream containing deionized water pumped with an Alltech liquid chromatography pump and a liquid surfactant (28% sodium laureth sulfate) is pumped with a 500D Isco syringe pump to form a HIPR emulsion.

The HIPR emulsion is conveyed to a second four-inch diameter Oakes mixer spinning at 540 rpm and diluted with a second aqueous phase stream containing deionized water pumped with an Alltech liquid chromatography pump. Next, the reduced volume fraction emulsion is combined with a cross-linking agent stream of 10% weight aqueous solution of ethylene diamine (EDA) pumped with a 500D Isco syringe pump.

Rate of oil phase stream: 34.4 g/min; rate of first aqueous stream: 4.0 g/min; Rate of surfactant stream: 1.8 g/min; Rate of second aqueous stream: 26 g/min; and rate of crosslinking agent: 8.6 g/min

### Example 3

A dispersion of core-shell mesocapsules is formed according to yet another embodiment of the present invention is prepared substantially the same as Example 2, except that the crosslinking agent is a 25% weight aqueous solution of lysine, pumped at a rate of 5.6 g/min.

### Example 4

A dispersion of core-shell mesocapsules is formed according to yet another embodiment of the present invention is prepared substantially the same as Example 2, except that no addition crosslinking agent is used, rather the reduced volume fraction emulsion is allowed to sit overnight. Isocyanate polymerizes in the presence of water. Rate of oil phase stream: 33.2 g/min; rate of first aqueous stream: 3.0 g/min; Rate of surfactant stream: 1.8 g/min; and Rate of second aqueous stream: 27 g/min.

### Example 5

Compositions according to Examples 1-4 were prepared substantially as described above and their particle sizes determined by conventional methods using a Coulter LS230 laser light scattering particle sizer. The particle size is defined by the volume average diameter, and reported in TABLE 1:

| | Particle size |
|---|---|
| Sample | (μm) |
| Example 1 | 1.07 |
| Example 2 | 0.85 |
| Example 3 | 0.89 |
| Example 4 | 0.52 |

Particle size of the mesocapsules was affected by the flow rates of the streams more than crosslinker in the case of Examples 2 and 3.

### Example 6 (Comparative)

Conventional core-shell particles are prepared by interfacial polymerization. 10.0 g avobenzone, 5.0 g homosalate, 5.0 g octocrylene, 2.0 g polymeric isocyanate (PAPI 27), 0.6 g laureth-23, 0.6 g ceteth-20 are heated to 60° C. and mixed until uniform. To this is added 1.2 g of a 28% aqueous sodium laureth sulfate solution and 13.8 g of deionized water. All the ingredients are mixed with a PowerGen 700D homogenizer (Fisher Scientific) for 60 seconds at 10,000 rotations per minute (rpm) to form mesocapsules. Particle size of the mesocapsules is measured using Coulter LS230 laser light scattering particle sizer and is defined by the volume average diameter. Conventional had a particle size of 2.48 μm.

Without being bound by theory, smaller particle size increases scattering and improves the UV absorber effectiveness, also promotes uptake in other actives. Accordingly, one advantage of the inventive process is that it is possible to create smaller particles. Advantageously, the inventive process offers controlled particle size encapsulated material at a reasonable cost.

1. A process for encapsulating a hydrophobic active in a core-shell mesocapsule comprising:
preparing a high internal phase ratio emulsion having a continuous aqueous phase and a dispersed oil phase comprising at least one hydrophobic active and one or more pre-polymers;
reducing the volume fraction of the high internal phase ratio emulsion below 0.74 with an aqueous phase stream, and then
forming a dispersion of core-shell mesocapsules containing hydrophobic active by either:
i) allowing the reduced volume fraction emulsion to sit for 12 hours when the pre-polymer is isocyanate; or
ii) contacting the reduced volume fraction emulsion with a third aqueous stream comprising a cross-linking agent.

2. The process of claim 1, wherein the step of reducing the volume fraction of the high internal phase ratio emulsion below 0.74 with an aqueous phase stream is performed within two hours of preparing the high internal phase ratio emulsion.

3. The process of claim 1, wherein the dispersion is 40% to 55% solids, preferably 45% to 50% solids.

4. The process of claim 1, wherein the high internal phase ratio emulsion has a dispersed oil phase volume fraction of greater than 0.74.

5. The process of claim 1, wherein the high internal phase ratio emulsion is 80% to 95% solids, preferably 85% to 90% solids.

6. The process of claim 1, wherein the step of preparing a high internal phase ratio emulsion comprises continuously merging in the presence of an emulsifying and stabilizing amount of a surfactant an aqueous phase stream and an oil phase stream.

7. The process of claim 1, wherein the one or more pre-polymers comprises a polyisocyanate selected from a group of 4,4′-diisocyanatodiphenylmethane, 2,4′-diisocyanatodiphenylmethane, p-phenylene diisocyanate, 2,6-toluene diisocyanate, polyphenyl polymethylene polyisocyanate, 1,3-bis(isocyanatomethyl)cyclohexane, 1,4-diisocyanatocyclohexane, hexamethylene diisocyanate, 1,5-naphthalene diisocyanate, 3,3′-dimethyl-4,4′-biphenyl diisocyanate, 4,4′-diisocyanatodicyclohexylmethane, 2,4′-diisocyanatodicyclohexylmethane, isophorone diisocyanate, or 2,4-toluene diisocyanate, or a combination thereof.

8. The process of claim 1, wherein the one or more pre-polymers is present in the oil phase at no more than 20% by volume.

9. The process of claim 1, wherein the personal care agent is a sun-screen agent selected from a group of paraminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide and zinc oxide, diethanolamine methoxycinnamate, digalloy trioleate, ethyl dihydroxypropyl PABA, glyceryl aminobenzoate, lawsone with dihydroxy acetone, red petrolatum and any combinations thereof.

10. The process of claim 1, wherein the cross-linking agent comprises a hydroxyl-containing or amine-containing compound.

11. The process of claim 1, wherein the polymeric shell comprises polyurea.

12. The process of claim 1, wherein the volume-average diameter of the core-shell mesocapsule is between 500 nanometers and 1500 nanometers.

13. A process for encapsulating a hydrophobic active in a core-shell mesocapsule comprising:
preparing a high internal phase ratio emulsion having a continuous aqueous phase and a dispersed oil phase comprising at least one sunscreen active and one or more isocyanate;
reducing the volume fraction of the high internal phase ratio emulsion below 0.74 with an aqueous phase stream, and then
forming a dispersion of polyurea mesocapsules containing sunscreen active by contacting the reduced volume fraction emulsion with a third aqueous stream comprising an amine.

14. Use of the dispersion of core-shell mesocapsules of claim 1 or 13 in a personal care composition without further processing.
